(19) United States
(12) Patent Application Publication
(10) Pub. No.: US 2011/0259347 A1
(43) Pub. Date: Oct. 27, 2011

(54) Orthopedic Disposable Bilateral Split Sheet

(76) Inventor: Frances Zurn, Nicholson, PA (US)

(21) Appl. No.: 13/092,117

(22) Filed: Apr. 21, 2011

Related U.S. Application Data
(60) Provisional application No. 61/326,824, filed on Apr. 22, 2010.

(51) Int. Cl. A61B 19/08

(52) U.S. Cl. 128/849

(57) ABSTRACT

A sterile surgical sheet comprising two parallel slits and bands of adhesive bonding for prepping a patient for bilateral knee replacement surgery and providing adequate shielding from an upper thigh tourniquet. The sheet is of one-piece construction and utilizes two parallel slits for placement around a patient's legs to maintain a sterile field and seal off a tourniquet area. Outer sections of material are wrapped around and over each leg from the outside, while an inner section between the slits is folded over the patient's groin and over the outer section wrapping. Bands of adhesive bonding along the slits secure the folded assembly against the patient's legs and ensure the sheet does not come unwrapped. This provides doctors and nurses with a bounded sterile region and uninhibited access to a patient's legs. The one-piece drape provides convenience for users and reduces effort when preparing a patient prior to surgery, eliminating the use of multiple, overlapping sheets that are prone to separation.

### CROSS REFERENCE TO RELATED APPLICATION

This application claims the benefit of U.S. Provisional Application No. 61/326,824 filed on Apr. 22, 2010, entitled “Orthopedic Disposable Bilateral Split Sheet.”

### BACKGROUND OF THE INVENTION

1. Field of the Invention

The present invention relates to surgical drapes and sterilized coverings in the surgical field. More specifically, the present invention relates a surgical drape with parallel leg slits for prepping patients for bilateral knee replacement surgery and providing adequate coverage for unsterile thigh tourniquets.

2. Description of the Prior Art

Surgical drapes are extensively used in the orthopedic field for isolating areas or portions of the body prior to surgery. The drapes are generally impermeable to fluids, and provide a sterile barrier between an area of interest and the rest of the patient. It is common to use several drapes to isolate large areas, either wrapping or otherwise attaching the drapes around a patient prior to the procedure. The drapes are typically general purpose, rectangular shaped sheets that are not designed to accommodate bilateral limbs of a human body. Efficiently prepping a patient and ensuring the wrapping remains securely in place during a procedure can be troublesome for these sheets.

Devices have been proposed in the prior art for solving this issue, most notably U.S. Pat. No. 4,569,341 to Morris and U.S. Pat. No. 4,479,492 to Singer. These patents disclose specifically designed drapes that are utilized to prep a patient prior to an orthopedic operation, particularly an operation on an extended limb or joint. While they are useful for isolating a limb, they do not incorporate material that prevents strikethrough of orthopedic instruments during surgery and do not incorporate the same design elements as the present invention. They also do not provide a structure that is capable of attaching to a patient and draping a leg tourniquet prior to leg operations.

The current method of draping a patient prior to a bilateral knee replacement surgery involves utilization of two split sheets or two standard drapes. The legs of the patient are lifted and wrapped with the plurality of sheets, one leg at a time. The sheets are wrapped and secured using more material than is necessary to ensure proper coverage and secure wrapping. Avoiding contamination with unsterilized areas of the body is also of primary concern, which further impedes the wrapping process and takes up more time, introducing unnecessary risks and delay.

The present invention eliminates the waste and improves efficiency by providing a single drape with a plurality of leg slits to drape around the legs of a patient and secure with attached adhesive boding. The wrapping is secured to the legs around the base of each split, creating a clear and stable boundary between sterilized and unsterile areas of the patient. The drape also provides coverage for unsterilized thigh tourniquets during a procedure. The drape material is a liquid impermeable material, as well as strikethrough resistant to prevent inadvertent puncturing of the draping during a surgical procedure.

In this regard the present invention substantially diverges in design elements and features, and is distinguishable from the prior art by providing a new and unique surgical drape that fulfills a need in the art. The present invention provides a new and improved split sheet device that has all of the advantages of the prior art and none of the disadvantages.

### SUMMARY OF THE INVENTION

In view of the foregoing disadvantages inherent in the known types of surgical split sheet drapes now present in the prior art, the present invention provides a new surgical split sheet drapes wherein the same can be utilized for providing convenience for the user when preparing a patient prior to a surgical procedure involving the legs in which a sterile field is required.

It is therefore an object of the present invention to provide a one-piece drape with a plurality of slits to accommodate the legs of a patient, providing a single sheet for wrapping the legs and creating a boundary between an area of interest and the rest of the unsterilized patient.

Another object of the present invention is to provide a split sheet that has adhesive bonding along the slits to provide a means to secure the sheet against the patient and to further secure the wrapping to itself during surgical preparation.

Yet another object of the present invention is to provide a sheet of material that is both liquid impermeable, hypoallergenic, sterile and strikethrough resistant.

Yet another object of the present invention is to provide a sheet specifically designed to accommodate bilateral knee replacement surgery and shield unsterilized thigh tourniquets.

Other objects, features and advantages of the present invention will become apparent from the following detailed description taken in conjunction with the accompanying drawings.

### BRIEF DESCRIPTIONS OF THE DRAWINGS

FIG. 1 shows a perspective view of the bilateral split sheet in its working position.

FIG. 2 an overhead view of the bilateral split sheet in its unfolded configuration.

### DETAILED DESCRIPTION OF THE INVENTION

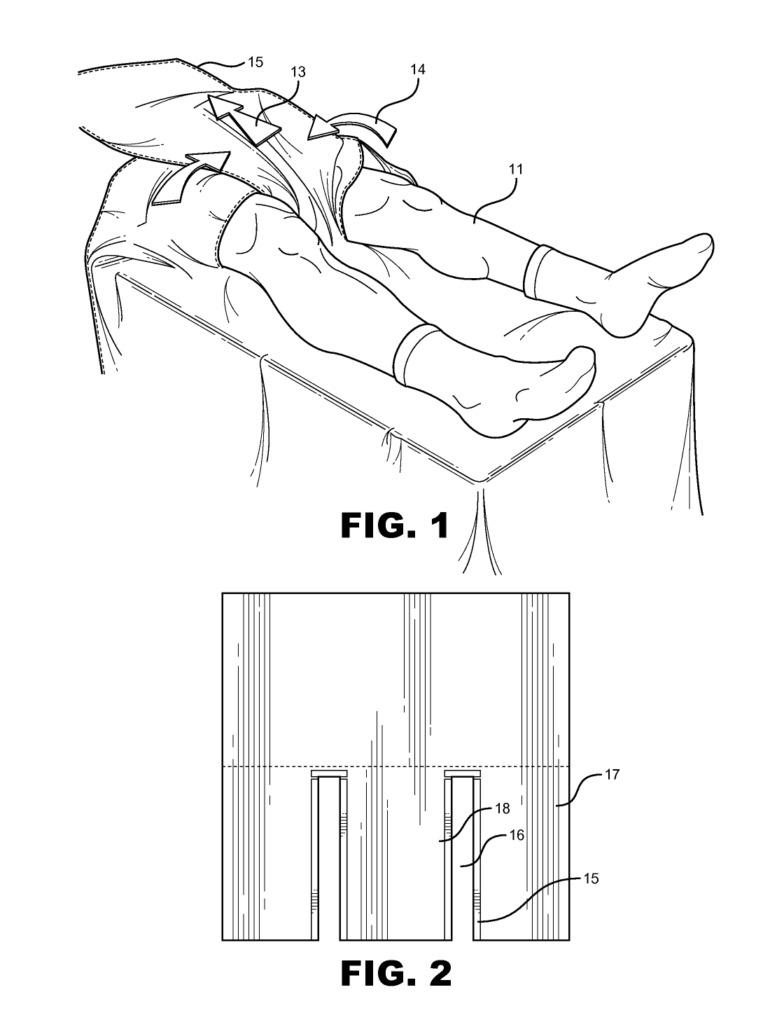

Referring now to FIG. 1, there is shown a perspective view of the bilateral split sheet in its working position. The sheet is used to bound a sterile area on the patient, as well as provide a sterile boundary from the operating table. The sheet comprises a large rectangular shape with two parallel slits for accommodating a user's legs 11. Each slit is bounded by three strips of covered adhesive material. The slits separate the drape into two outer regions of draping, which are folded around and over 14 the patient's legs 11 toward the groin region. Between the slits is an inner region of draping 15, which is placed between the user's legs 11 and folded over the groin area 13. This region of draping is placed over the outer region draping and secured thereto. The outer regions of draping are adhered to the patient's legs 11 using the adhesive bonding, creating a clear boundary between the sterile and unsterile fields. The inner region is adhered to the outer region wrapping after application.

Referring now to FIG. 2, there is shown an overhead view of the bilateral split sheet, in its expanded configuration prior to use. The sheet comprises two parallel slits 16 that accommodate a patient's legs therein, along with strips of adhesive bonding 15 lining each slit. The slits 16 create two outer regions 17 of draping and an inner region 18. The outer region 17 wraps around the outside of the user's legs and over the groin area, followed by the inner region 18, which is wrapped over the groin and the wrapped outer regions 17.

In use a patient is prepared for a leg operation, such as a bilateral knee replacement or similar procedure, using the split sheet to drape the unsterilized regions of the patient and create a stable boundary therefrom. The sheet is designed to be used in the draping process for sealing off tourniquets, providing for a sterile field, and ensuring there is no strike-through during the procedure. The use of multiple drapes is eliminated by the singular split sheet, while facilitating a more efficient draping process. Orthopedic teams may benefit from this sheet by providing an easier, more cost effective method of draping for knees and leg surgeries

When applying the sheet to a patient, the patient is first placed on an operating table in a supine position. His or her legs are lifted from the table as the slits of the sheet engage the backside of each leg. The outer regions of draping are wrapped around the leg as the adhesive bonding is pressed against the patient's skin to create a secure boundary. Once the outer regions have been applied, the legs of the patient can be placed back onto the table and onto the homogeneous region of the sheet. The inner region is then wrapped over the groin and adhered to the already applied wrapping. Once the sheet is in place, a boundary between unsterile regions of the body and the operating table is created.

The sheet comprises of a liquid impermeable, double reinforced, latex-free, flame retardant material, similar to the drapes currently used in many operating rooms. The drape may be approximately 120″ long and 98″ wide, although it is not intended to restrict the sheet to these exact dimensions. The sheet may be used independently of other drapes, eliminating the need for a second drape in the preparation process. The inner and outer regions of the drape include adhesive bonding that is covered prior to use and exposed as the sheet is applied to the patient. In this manner, the drape adheres to itself to seal off the unsterile areas. This allows the drape to accommodate both extremities simultaneously.

Three strips of adhesive material bound each slit, and are covered by a removable layer prior to preparing a patient. The covering for each adhesive slit is removed as the slits are wrapped around the patient's legs and the adhesive is attached to the user.

Thigh tourniquets are common for bilateral knee replacement surgery, which comprise compression straps high along the patient's legs just below the groin area. The tourniquets are unsterile structures that must be avoided during the procedure. The present invention provides sufficient material to provide covering for these structures in order to prevent inadvertent contact and contamination with sterile equipment or practitioners.

The present invention may also be used in other surgical procedures involving a patient's legs, including femoral popiteal bypass surgery. The outer regions of the drape may be adhered to the outside of each leg or in any other configuration deemed necessary by the preparing nurse or practitioner.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A surgical draping for bilateral knee replacement surgery, comprising:
A rectangular sheet of liquid impermeable, strikethrough resistant material;
a plurality of parallel slits extending from one edge of said sheet;
strips of adhesive bonding lining each said slit.

2) A method of draping a tourniquet region of a patient prior to bilateral knee replacement surgery, comprising the steps of:
placing a patient with thigh tourniquets around each leg on an operating table in a supine position,
unfolding a bilateral split sheet and removing coverings from adhesive bonding surrounding each parallel slit of said split sheet,
lifting said legs from said table and placing each slit around each leg, securing said slits against said legs using said adhesive boding,
draping a region outside of said slits around said legs and draping a region between said slits over said patient's groin area, providing draping for said tourniquets.
